(19) United States
(12) Patent Application Publication
(10) Pub. No.: US 2024/0408389 A1
(43) Pub. Date: Dec. 12, 2024

(54) SYSTEMS AND METHODS FOR ELECTRICAL MUSCLE STIMULATION

(71) Applicant: Rehabtronics Inc., Edmonton (CA)

(72) Inventors: Rahul SAMANT, Edmonton (CA); Kanyamashokoro CESAR, Edmonton (CA); Mustafa Kaya, Edmonton (CA)

(73) Assignee: Rehabtronics Inc., Edmonton, AB (CA)

(21) Appl. No.: 18/702,527

(22) PCT Filed: Oct. 19, 2022
(86) PCT No.: PCT/CA2022/051541

§ 371 Date: Apr. 18, 2024

Related U.S. Application Data
(60) Provisional application No. 63/257,944, filed on Oct. 20, 2021.

(51) Int. Cl. A61N 1/36; A61N 1/04; H02J 50/10

(52) U.S. Cl. CPC A61N 1/36003; A61N 1/0452; A61N 1/0492; A61N 1/36034; H02J 50/10

(57) ABSTRACT

An electrical stimulation system that includes a stimulator including a generator and a controller, the generator is configured to generate an electrical current and the controller is configured to receive a user input for activating the generator, an electrode including a flexible body and one or more electrode pads positioned along the flexible body, and a connector configured to extend through the flexible body and electrically couple with the stimulator by inductive coupling, wherein the connector is configured to electrically couple the generator to the one or more electrode pads in response to the stimulator being positioned within a predefined distance to the connector, such that the controller is configured to wirelessly transmit the electrical current from the generator to the one or more electrode pads via the connector in response to the user input.

### CROSS-REFERENCE TO RELATED APPLICATIONS

This patent application claims the benefit of priority to U.S. Provisional Patent Application No. 63/257,955, filed on Oct. 20, 2021, the entirety of which is incorporated by reference herein.

### FIELD OF DISCLOSURE

Aspects of the present disclosure relate generally to systems, assemblies, and methods for providing electrical stimulation. More specifically, particular embodiments of the present disclosure relate to systems and methods for using an electrical stimulation system to generate and deliver electrical stimulation to a body of a user.

### Introduction

Electrical muscle stimulation (EMS) includes eliciting muscle contractions using electrical impulses. Electrical impulses may be generated by a stimulating device, and delivered to an individual's target muscles through electrodes placed near the muscles. EMS technology has not gained mainstream adoption beyond certain medical, therapeutic, and specialized uses, at least in part because EMS systems may be relatively large, unwieldy, non-portable, and complicated to use. EMS systems also may not connect with modern, everyday consumer technology, such as mobile phones, tablets, and/or wearable devices (e.g., smart watches). The present disclosure addresses needs that remain generally unmet by EMS technology.

EMS systems may include a generator, such as a waveform generator, and a controller that may be controlled by software or hardware (e.g., a button, a knob, a touchscreen, a dial, etc.). The waveform generator may be connected to one or more electrodes via a series of wires, and the electrodes may have an adhesive side for contacting and sticking to a user's body, thereby securing the EMS system to the user. One or more devices of the EMS system, such as the electrodes, may be a single-use component or a reusable component.

The background description provided herein is for the purpose of generally presenting the context of this disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for generating and delivering electrical stimulation to a user's body, such as to one or more target muscles on the body. Each of the aspects disclosed herein may include one or more of the features described in connection with any of the other disclosed aspects.

According to one example of the present disclosure, an electrical stimulation system includes a stimulator including a generator and a controller, the generator is configured to generate an electrical current and the controller is configured to receive a user input for activating the generator; an electrode including a flexible body and one or more electrode pads positioned along the flexible body; and a connector configured to extend through the flexible body and electrically couple with the stimulator by inductive coupling, wherein the connector is configured to electrically couple the generator to the one or more electrode pads in response to the stimulator being positioned within a predefined distance to the connector, such that the controller is configured to wirelessly transmit the electrical current from the generator to the one or more electrode pads via the connector in response to the user input.

In some aspects of the present disclosure, the predefined distance includes about 1 centimeter to about 30 centimeters. The inductive coupling between the connector and the stimulator includes an electromagnetic connection. The connector is configured to wirelessly transmit the electrical current from the generator to the connector, and the connector is configured to distribute the electrical current to the one or more electrode pads. The stimulator includes one or more magnets and the connector includes one or more corresponding magnets that are configured to mate with the one or more magnets of the stimulator, thereby magnetically coupling the stimulator to the connector. The controller is configured to determine a speed, a duration, an intensity, or a frequency of the electrical current generated by the generator. The flexible body includes a main portion and one or more legs extending outwardly from the main portion, wherein the connector is configured to extend through the flexible body at the main portion, and each of the one or more legs includes at least one of the one or more electrode pads. The one or more electrode pads are selectively coupled to the flexible body, and configured to attach the electrode to a body of a user. The one or more electrode pads define an adhesive interface of the flexible body that is configured to couple the electrode to the body of the user. The electrode includes one or more wires enclosed or embedded within the flexible body, the one or more wires are configured to electrically couple the one or more electrode pads with the connector. The flexible body includes a connection interface defining one or more openings, the connection interface is configured to receive at least a portion of the connector through the one or more openings.

In some aspects of the present disclosure, the connector has a housing that is configured to receive at least a portion of the flexible body, thereby coupling the electrode to the connector. The connector includes one or more sensors configured to detect one or more operating characteristics of the stimulator or the electrode. The controller is configured to activate the generator in response to the one or more sensors detecting an electrical connection between the stimulator and the electrode.

The stimulator includes a housing formed of a biocompatible material, a water-resistant material, a shatter-resistant material, or a shock-resistant material. The stimulator includes an indicator positioned along an exterior surface of the housing, the indicator is configured to generate a user feedback indicative of an operation status of the stimulator or the electrode. The stimulator includes at least one rechargeable battery that is configured to supply electrical power to one or more of the generator and the controller. The stimulator is communicatively coupled to at least one external electronic device, and the controller is configured to receive the user input for operating the generator from the at least one external electronic device. The generator is configured to generate an electrical waveform over a wide range of signals, and to convert the electrical waveform to one or more pulses of the electrical current. The stimulator includes one or more sensors configured to detect an electrical connection between the stimulator and the electrode, wherein the controller is configured to automatically activate the generator in response to the one or more sensors detecting the electrical connection.

According to another example of the present disclosure, a method for operating an electrical stimulation system that includes a stimulator, a connector, and an electrode, wherein the stimulator is electromagnetically coupled to the connector and the electrode is at least partially received within an interior of the connector, such that the connector is configured to electrically couple the stimulator to the electrode, the method comprising attaching the electrode to a body of a user such that the electrode is in contact with the body, activating the stimulator to generate an electrical pulse that is transferred to the connector, and delivering the electrical pulse from the stimulator to the electrode via the connector, thereby stimulating a portion of the body that is in direct contact with the electrode.

In some aspects of the present disclosure, the electrical stimulation system is communicatively coupled with an external electronic device, and the stimulator is configured to receive a user input from the external electronic device for activating the stimulator. The external electronic device includes a software application that is communicatively coupled with the stimulator, such that the user input is generated at the software application and transmitted to the stimulator. The user input defines one or more parameters of the electrical pulse including a speed, a duration, an intensity, or a frequency of the electrical pulse generated by the generator. A plurality of electrical stimulation systems are communicatively coupled to the external electronic device. In some aspects of the present disclosure, the at least one sensor that is configured to detect an electrical connection between the stimulator and the electrode, the method comprises activating the stimulator automatically in response to the at least one sensor detecting the electrical connection between the stimulator and the electrode. The stimulator includes one or more conductive pins configured to electrically couple with a control board of the connector, such that the control board is in electrical communication with the generator and the controller. The connector includes an electrode plate that is in electrical communication with the control board and the one or more electrode pads, the electrode plate is configured to receive the one or more conductive pins, thereby electrically coupling the electrode to the stimulator via the connector. The connector is configured to transmit the electrical current generated by the generator to the one or more electrode pads via a connection between the electrode plate and each of the one or more conductive pins and the one or more electrode pads.

According to another example of the present disclosure, a computer-implemented method for operating an electrical stimulation system, the electrical stimulation system including a stimulator, a connector, and an electrode, wherein the stimulator includes a conductive pin, the electrode includes an electrode pad, and the connector includes a power plate that is configured to electrically couple the conductive pin with the electrode pad, the method comprising connecting a user device with the stimulator, such that the user device is in communication with the stimulator, generating an electrical current with the stimulator in response to the stimulator receiving at least one input from the user device wherein the electrical current is transmitted to the connector via connection between the conductive pin and the power plate, and delivering the electrical current from the electrode pad in response to the electrode receiving the electrical current from the connector, wherein the electrical current is transmitted to the electrode via connection between the power plate and the electrode pad. In some aspects of the present disclosure, the user device is wirelessly connected to the stimulator via Bluetooth Low Energy or Wi-Fi.

It is to be understood that both the foregoing general description and the following detailed description are exemplary and explanatory only, and are not restrictive of the disclosed embodiments, as claimed. As used herein, the terms “includes,” “including,” “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” For such terms, and for the terms “for example” and “such as,” and grammatical equivalences thereof, the phrase “and without limitation” is understood to follow unless explicitly stated otherwise. As used herein, the terms “about” and “approximately” are meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise. Moreover, in the claims, values, limits, and/or other ranges mean the value, limit, and/or range +/−10%.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

FIG. 1 depicts an exemplary electrical stimulation system including a stimulator device and an electrode device, according to one or more embodiments.

FIG. 2 depicts the stimulator device of the electrical stimulation system of FIG. 1 in a disassembled state, according to one or more embodiments.

FIG. 3 depicts the stimulator device of FIG. 2, according to one or more embodiments.

FIG. 4 depicts an exemplary connector device of the electrical stimulation system of FIG. 1, according to one or more embodiments.

FIG. 5 depicts the connector device of FIG. 4 in a disassembled state, according to one or more embodiments.

FIG. 6 depicts the electrode device of the electrical stimulation system of FIG. 1, according to one or more embodiments.

FIG. 7 depicts an exploded view of the electrode device of FIG. 6, according to one or more embodiments.

FIG. 8 depicts an exploded view of the electrode device of FIG. 6 and the connector device of FIG. 4, according to one or more embodiments.

FIG. 9 depicts the electrical stimulation system of FIG. 1 in a disassembled state, according to one or more embodiments.

FIG. 10 depicts a connection interface between the stimulator device of FIG. 2 and the connector device of FIG. 4, according to one or more embodiments.

FIG. 11 depicts a connection interface between an exemplary charger device and the stimulator device of FIG. 2, according to one or more embodiments.

FIG. 12 depicts an exemplary case for housing one or more devices of the electrical stimulation system of FIG. 1, according to one or more embodiments.

FIGS. 13-16 depict exemplary implementations of the electrical stimulation system of FIG. 1, according to one or more embodiments.

### DETAILED DESCRIPTION

Embodiments of this disclosure relate to electrical muscle stimulation (EMS) systems, devices, assemblies, and methods for eliciting electrical muscle contractions using electrical impulses. The EMS systems described herein may be utilized for various suitable uses, such as, for example, as a rehabilitation tool, an injury-preventive tool for immobilized patients, a strength-training tool for healthy individuals to evaluate neuromuscular function, a post-exercise recovery tool, and more.

The EMS systems described herein may include one or more of a combined generator and controller (collectively referred to herein as a “stimulator device”), one or more electrodes (collectively referred to herein as an “electrode device”), and/or a connector device communicatively coupling the stimulator device and the electrode device. The stimulator device and electrode device may be connected to one another (e.g., via the connector device) to assemble the EMS system. The EMS system may be an electrical muscle stimulation system, e.g., a transcutaneous electrical nerve stimulation (TENS) unit. The EMS system may be operable via a software application that may be run on, e.g., a computing device, such as a phone, a tablet, a personal computer, a wearable device, or other electronic device. The EMS system may interact with one or more computing devices via any suitable wireless connection, such as via a Bluetooth connection (e.g., Bluetooth Low Energy (BLE), Bluetooth 4.0, or Bluetooth SMART), Wi-Fi, or other data connection. Also disclosed herein are EMS system kits or cases for housing the one or more devices of the EMS system, including, for example, one or more stimulator devices, one or more electrode devices, one or more charging devices, and more. Also disclosed herein are methods of using EMS devices, systems, and kits.

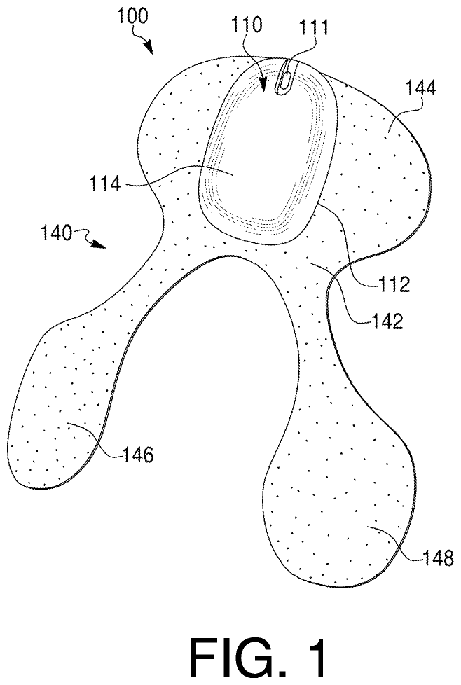

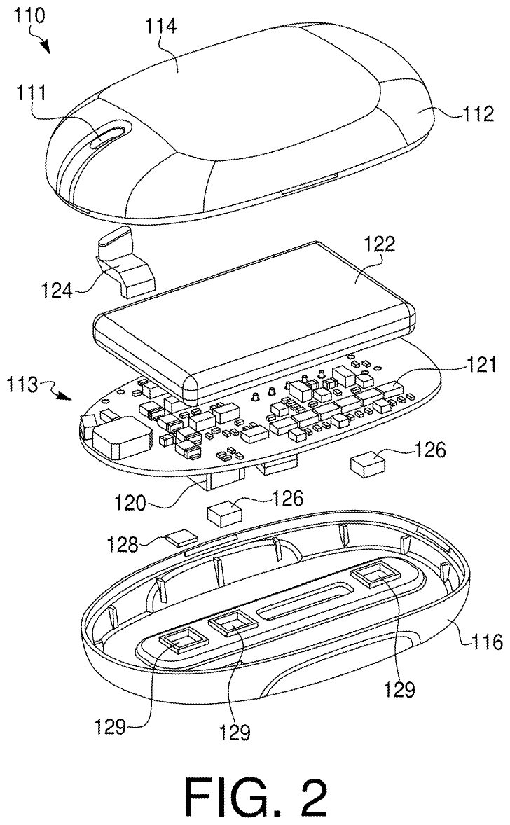

As shown in FIG. 1, an exemplary EMS system 100 disclosed herein may include a stimulator device 110 and an electrode device 140. Stimulator device 110 may include a housing 112 with a first wall 114 and a second wall 116 (see FIG. 2). Housing 112 may include one or more user interfaces and/or indicators operable to display an operating state of stimulator device 110. In the example, housing 112 may include an indicator 111 positioned first wall 114, and indicator 111 may define a user interface configured to display information to a user of EMS system 100.

As described in detail herein, stimulator device 110 may include one or more components, such as, for example, a generator (e.g., a waveform generator), a controller, and a power source (e.g., a rechargeable battery) housed within housing 112 (see FIG. 2). Stimulator device 110 and electrode device 140 may be configured to operate while in physical proximity relative to each other, such as through inductive coupling. In some embodiments, housing 112 of stimulator device 110 may be sized and/or shaped generally smaller than electrode device 140 (e.g., approximately palm-sized or smaller), such that stimulator device 110 may be coupled to a portion of electrode device 140. In an exemplary embodiment, housing 112 of stimulator device 110 may have a length of about 6 centimeters, a width of about 4 centimeters, and a height of about 1.5 centimeters. Stimulator device 110 may be connectable to one or more electronic devices (e.g., a mobile phone, a tablet, a personal computer, a cloud computer system, a wearable device, etc.) for controlling an operation of EMS system 100. Stimulator device 110 may be connected to the electronic device(s) by a wireless connection (e.g., Wi-Fi, data connections, Bluetooth, etc.) or a wired connection (e.g., USB, micro-USB, etc.).

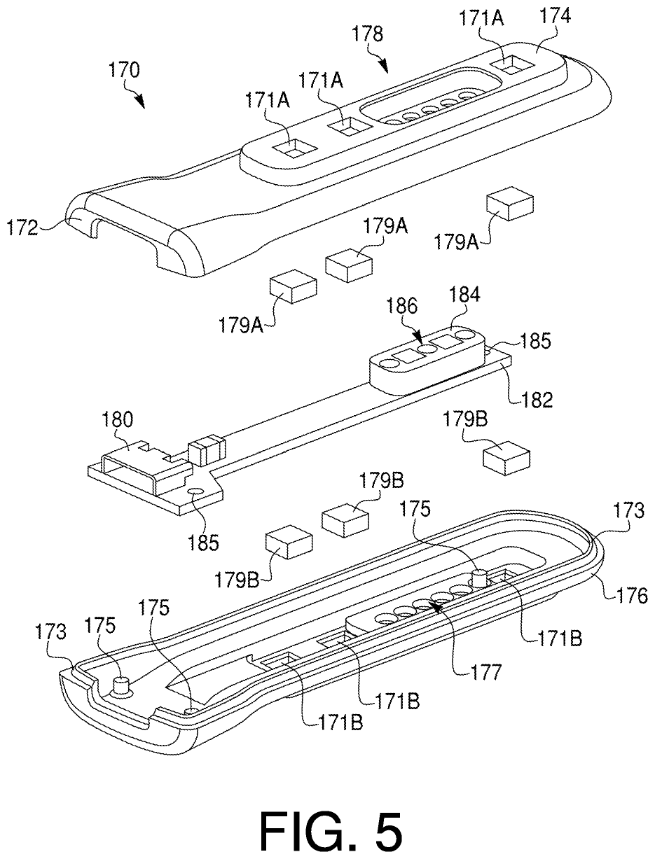

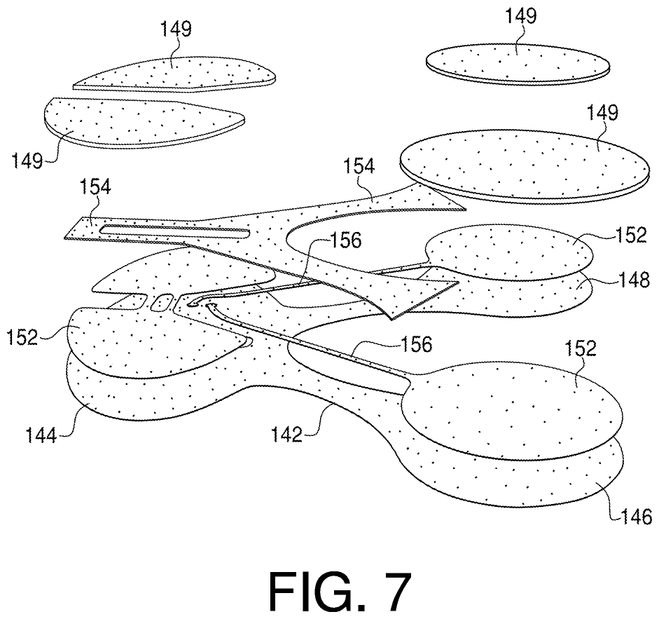

Still referring to FIG. 1, electrode device 140 may include a flexible body 142 having various suitable sizes, shapes, and/or configurations for facilitating attachment to a user's body. In some embodiments, flexible body 142 may have one or more ends and/or portions, and flexible body 142 may be selectively deformable, such as relative to a contour of the user's body. As such, flexible body 142 may enable electrode device 140 to flex, move, and/or otherwise change shape in accordance with an attachment surface (e.g., a user's skin surface) without becoming inoperable and/or causing the user discomfort. In the example, flexible body 142 may include at least a first end 144, a second end 146, and a third end 148 that collectively define a general V-shaped configuration of flexible body 142. It should be appreciated that flexible body 142 may include a main portion (e.g., first end 144) and one or more arms and/or legs (e.g., second end 146, third end 148) extending outwardly from the main portion. The main portion of flexible body 142 may be configured to receive a connector device and stimulator device 110 (see FIGS. 4-6 and 8-10), as described in more detail below, and the one or more arms and/or legs may be configured to receive one or more electrode pads (see FIGS. 6-8). It should be appreciated that electrode device 140 may include additional and/or fewer ends, portions, arms/legs, and/or components than those shown and described herein without departing from a scope of this disclosure (see FIG. 14).

FIG. 2 depicts housing 112 of stimulator device 110 in a disassembled state. Stimulator device 110 may include a stimulator mechanism 113 and a battery 122 disposed between first wall 114 and second wall 116 of housing 112. Housing 112 may be configured to enclose and protect the components of stimulator device 110 from damage, dust, moisture, and/or other environmental elements. It should be appreciated that housing 112 may have various suitable sizes and/or shapes. In some embodiments, housing 112 may have an ergonomic profile, suitable for being hand-held by a user. In some embodiments, housing 112 may have a generally ovoid profile. Housing 112, and particularly first wall 114 and second wall 116, and/or its contents, may be made of materials that are biocompatible, waterproof or water-resistant, shatterproof or shatter-resistant, and/or shockproof or shock-resistant. In some embodiments, housing 112 may be made from plastic.

In some embodiments, housing 112 may be made from two approximately dome-shaped walls that form two halves of a shell structure of stimulator device 110, e.g., first wall 114 and second wall 116. In the example, the dome-shape of housing 112 may be operable to distribute a load throughout the external walls of the shell (e.g., first wall 114, second wall 116), thereby inhibiting an application of excessive load at a single point along housing 112. First wall 114 and second wall 116 may be joined together by various suitable mechanisms, such as, for example, heat sealing, an adhesive (e.g., glue), welding, one or more screws, one or more clips, and/or other fastening mechanisms.

In some embodiments, housing 112 may include various internal features configured to secure and hold the internal components therein, e.g., stimulator mechanism 113 and/or battery 122. For example, in some embodiments, housing 112 may include an internal ribbing structure that may simultaneously hold battery 122 and provide structure to housing 112. In some embodiments, at least one wall of housing 112 (e.g., first wall 114, second wall 116) may include an indentation and/or interface to facilitate connection with a connector device, as discussed in detail below. In some embodiments, housing 112 may include one or more recesses (e.g., on first wall 114, second wall 116, and/or the sides of housing 112) to allow for a user to grip, attach, and/or remove stimulator device 110 from one or more other components of EMS system 100.

As further depicted in FIG. 2, stimulator mechanism 113 may include a generator 120 and a controller 121. Generator 120 may include a waveform generator configured to generate an alternating electrical current or pulse (e.g., for delivering an alternating biphasic high-frequency pulse series), a continuous electrical current or pulse, a pulsating electrical current or pulse (e.g., for delivering intermittent pulses), and/or any combination thereof. In the example, controller 121 and generator 120 may be optimized for small size, high efficiency power usage, and/or high stimulation signal intensity and variety performance, to maximize a mobility, a portability, and a performance of stimulator device 110. Controller 121 may be configured to receive instructions from one or more external electronic devices communicatively coupled to stimulator device 110, such as, for example, a software application or a user interface of a remote device (not shown), which may receive inputs from a user for operating EMS system 100. Controller 121 and/or generator 120 may be configured to be wirelessly coupled and/or controlled by one or more aspects of EMS system 100. In other embodiments, controller 121 and/or generator 120 may be wirelessly coupled and/or controlled by one or more other devices or systems (e.g., an external electronic device).

For example, controller 121 may be configured to receive instructions as to one or more operational parameters of stimulator device 110, including but not limited to, a speed, a frequency, and/or an intensity of electrical stimulation generated by generator 120, such as from a software application and/or a user interface of the remote device. Controller 121 may be further configured to interpret such instructions into a series of waveforms, and thereby send instructions to generate the waveforms to generator 120. Generator 120 may be configured to receive the instructions from controller 121, including instructions to initiate generating waveforms, cease generating waveforms, and/or change a speed, an intensity, and/or a frequency of waveforms. It should be appreciated that the waveforms may be converted to electrical pulses, e.g., series of electrical current, which may be output from stimulator device 110 and transferred to electrode device 140.

Still referring to FIG. 2, stimulator device 110 may be powered via battery 122, and/or may be powered via a remote device (not shown), such as via a wired connection to (e.g., an A/C cord, a USB connection, etc.). Battery 122 may include one or more replaceable or non-replaceable batteries, and may be disposed inside housing 112 between first wall 114 and second wall 116. In some embodiments, battery 122 may include one or more rechargeable batteries, such as lithium ion batteries. Particularly, battery 122 may include a rechargeable, non-replaceable, lithium ion/polymer battery with an integrated protection control module, with a capacity not exceeding 400 milliamp hours (mAh). In this instance, battery 122 may be charged via a wired connection or a wireless charging protocol, as described in detail below.

In some embodiments, housing 112 may be sized and/or shaped to provide a spatial clearance therein for instances of bloating by battery 122. For example, a spatial clearance within housing 112 may include an area positioned around battery 122 that may be equal to about 10% of the volume of battery 122. Where stimulator device 110 has one or more rechargeable batteries 122, a compatible charger and/or charging cable may be used to charge or recharge batteries 122. In some embodiments, battery 122 may be capable of receiving charge from a fully depleted state to a fully charged state within a predefined duration, such as about 6 hours or less, about 5 hours or less, about 4 hours or less, or about 3 hours or less. Stimulator device 110, and particularly housing 112, may have one or more ports through which a charging cable or charger may be electrically connected to one or more components of stimulator device 110, e.g., controller 121 and/or battery 122. For example, in some embodiments, stimulator device 110 may have a USB charging port (not shown) along housing 112 through which a USB cable may be connected to stimulator device 110 for charging battery 122.

Stimulator device 110 may include one or more safety features for controlling an electrical output from generator 120. For example, stimulator device 110 may include voltage limiting hardware to limit an output voltage from generator 120 to a maximum threshold (e.g., about 120 Volts) in single-fault conditions. As another example, stimulator device 110 may include a slow blow fuse with a current rating (e.g., 2A rating), which may be electrically connected to battery 122. The slow bow fuse may be configured to absorb and/or carry a temporary surge current (i.e., overload) that exceeds the current rating. Stimulator device 110 may include various other features, such as, for example, an on-off switch/actuator (not shown) for selectively actuating stimulator device 110.

As described above, indicator 111 may define a user interface that is configured to display information to a user of EMS system 100, such as an operational state of stimulator device 110 (e.g., an active state, an inactive state, an idle state, etc.). In some embodiments, indicator 111 may include a light-emitting diode (LED) that is configured to emit a light of one more characteristics (e.g., a color, a pattern, an intensity, etc.). Stimulator device 110 may include an indicator guide 124 disposed between indicator 111 and battery 122. Indicator guide 124 may be configured to electrically couple indicator 111 to battery 122, thereby powering indicator 111. In some embodiments, indicator 111 may be configured to display information indicative of an amount of electrical power and/or charge remaining in battery 122. In other embodiments, indicator 111 and/or indicator guide 124 may be omitted entirely.

Still referring to FIG. 2, in some embodiments, stimulator device 110 may include one or more sensors (not shown) disposed within and/or along an exterior of housing 112. In the example, the one or more sensors may include Hall effect sensors configured to detect an external electrical connection to stimulator device 110, such as by an external electronic device. In some embodiments, the one or more sensors may be configured to automatically activate stimulator device 110 upon detecting an electrical connection between stimulator device 110 and one or more external electronic devices, such as, for example, a charger or electrode device 140, as described in detail herein.

Although not shown, in some embodiments stimulator device 110 may include one or more markings or labels along an exterior of housing 112, such as along first wall 114 and/or second wall 116. The one or more markings may display information indicative of the contents, components, and/or power capabilities of stimulator device 110. For example, the one or more markings may include a lot number label, a label for “non-ionizing radiation” (IEC 60417-5140 (2003-04)), a registered Federal Communications Commission (FCC) identification number label, a Waste from Electrical and Electronic Equipment (WEEE) symbol label, an Ingress Protection (IP) rating label, an identification and/or revision label, and more.

Still referring to FIG. 2, stimulator device 110 may include one or more first magnets 126 and one or more second magnets 128 disposed within housing 112. The one or more first magnets 126 and second magnets 128 may be positioned along various interior surfaces and/or locations within housing 112. As described herein, first magnet(s) 126 and second magnet(s) 128 may be collectively configured to couple stimulator device 110 to one or more devices of EMS system 100. In some embodiments, each of the one or more first magnets 126 may have the same or different magnetic strengths relative to one another. In the example, the one or more first magnets 126 may have a greater strength and/or size relative to the one or more second magnets 128. By including first magnets 126 and second magnet 128 of differing relative strengths, a magnetic connection between stimulator device 110 and one or more devices of EMS system 100 may be enhanced, such as, for example, to a connector device 170 (see FIG. 4). Further, including at least second magnet 128 of a lesser relative magnetic strength than first magnets 126 may allow for easier decoupling of stimulator device 110 from said devices at a location of stimulator device 110 adjacent to second magnet 128 relative to corresponding locations of first magnets 126.

In the example, stimulator device 110 may include a pair of first magnets 126 and at least one second magnet 128, and second wall 116 of housing 112 may include a corresponding cavity 129 along an interior surface of second wall 116 for receiving each of the pair of first magnets 126 and the at least one second magnet 128. Although stimulator device 110 is shown and described herein as having a pair of first magnets 126 and one second magnet 128, it should be appreciated that simulator device 110 may include additional and/or fewer first magnets 126 and/or second magnets 128 without departing from a scope of this disclosure. As described in detail herein, second wall 116 may define an interface for coupling stimulator device 110 to one or more devices of EMS system 100.

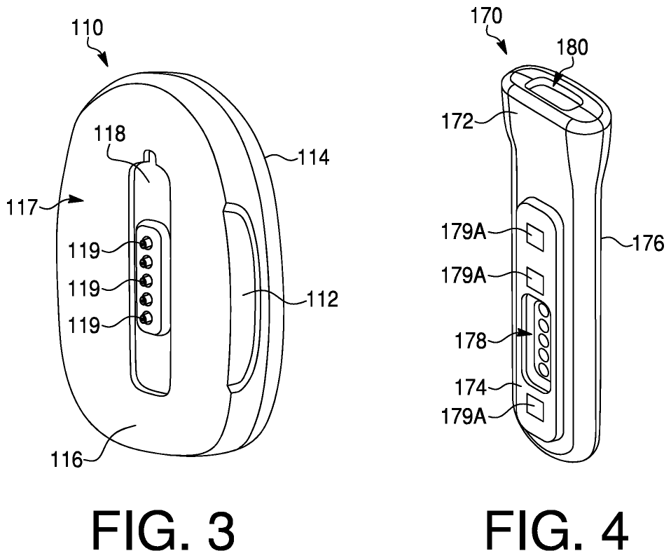

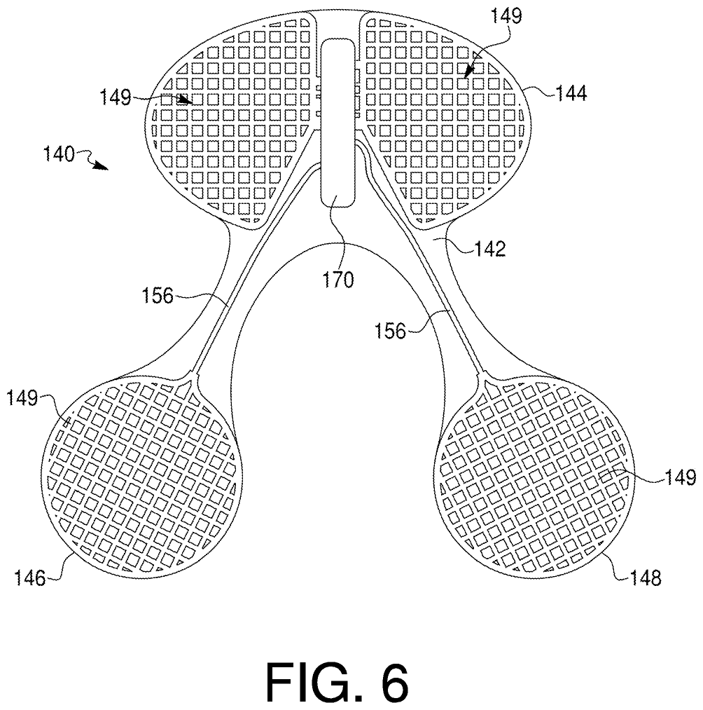

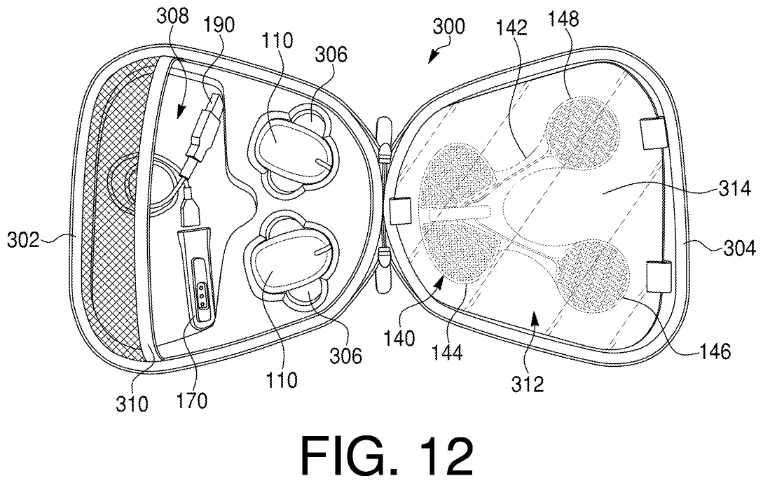

As depicted in FIG. 3, second wall 116 may include a bottom interface 117 having a recess 118 (e.g., an indentation) and one or more pins 119 disposed within recess 118. Recess 118 may be sized, shaped, and/or otherwise configured to mate with a corresponding feature of one more devices of EMS system 100 to facilitate a connection between stimulator device 110 and said device(s) (see FIG. 11). In the example, the one or more cavities 129 disposed within housing 112 (for receiving magnets 126, 128) may be aligned with recess 118, such that recess 118 may define a magnetic plate along bottom interface 117 for magnetically coupling housing 112 to one or more devices of EMS system 100. The one or more pins 119 may include a plurality of conductive prongs (e.g., pogo-pins) configured to electrically couple one or more components of stimulator device 110 to the device, and particularly one of more of stimulator mechanism 113 (e.g., generator 120, controller 121) and/or battery 122. For example, the device may include a connector device 170 (see FIGS. 4-5) and/or a charging device 200 of EMS system 100 (see FIG. 11).

Referring now to FIG. 4, connector device 170 may include a housing 172 having a first wall 174 and a second wall 176. Connector device 170 may include a first connection interface 178 disposed along first wall 174 and a charge port 180 positioned at a junction between the pair of walls 174, 176. First connection interface 178 may include one or more apertures for facilitating an electrical connection (e.g. electromagnetic) between connector device 170 and stimulator device 110 by receiving pins 119, as described further herein. In the example, connector device 170 may further include one or more first magnets 179A positioned along an exterior surface of first wall 174, and particularly received within one or more first cavities 171A on first wall 174. It should be appreciated that the quantity of first magnets 179A on connector device 170 may correspond to the quantity of magnets 126, 128 in stimulator device 110. Accordingly, first magnets 179A may be configured to facilitate a connection between connector device 170 and stimulator device 110, such as an electromagnetic connection. Stated differently, bottom interface 117 of stimulator device 110 may be configured to interface with first wall 174 of connector device 170. In this instance, the one or more first magnets 179A of connector device 170 may interact with first magnets 126 and second magnets 128, thereby magnetically coupling stimulator device 110 and connector device 170 to one another.

Upon coupling connector device 170 to stimulator device 110, first connection interface 178 may align with and receive the plurality of pins 119 on bottom interface 117, thereby electrically coupling one or more components of connector device 170 (e.g., a circuit board) to one or more components of stimulator device 110 (e.g., stimulator mechanism 113). In the example, connector device 170 may be electrically coupled to stimulator device 110 by inductive and/or electromagnetic coupling, such that stimulator device 110 may be configured to provide wireless inductive and/or magnetic resonant power transfer and communication with connector device 170 and/or directly with electrode device 140. As described herein, upon coupling connector device 170 to each of stimulator device 110 and electrode device 140, connector device 170 may be configured to electrically transmit electrical current generated by stimulator device 110 to electrode device 140. In further embodiments, connector device 170 may be configured to electrically couple stimulator device 110 to an external electronic device (not shown) via charge port 180. For example, charge port 180 may include a cable port (e.g., a USB charging port) that is configured to receive a cord or cable (e.g., a USB cable) of the external electronic device. In this instance, battery 122 may receive electrical power from the external electronic device via a connection of stimulator device 110 to connector device 170 at first connection interface 178 and of the external device to connector device 170 at charge port 180.

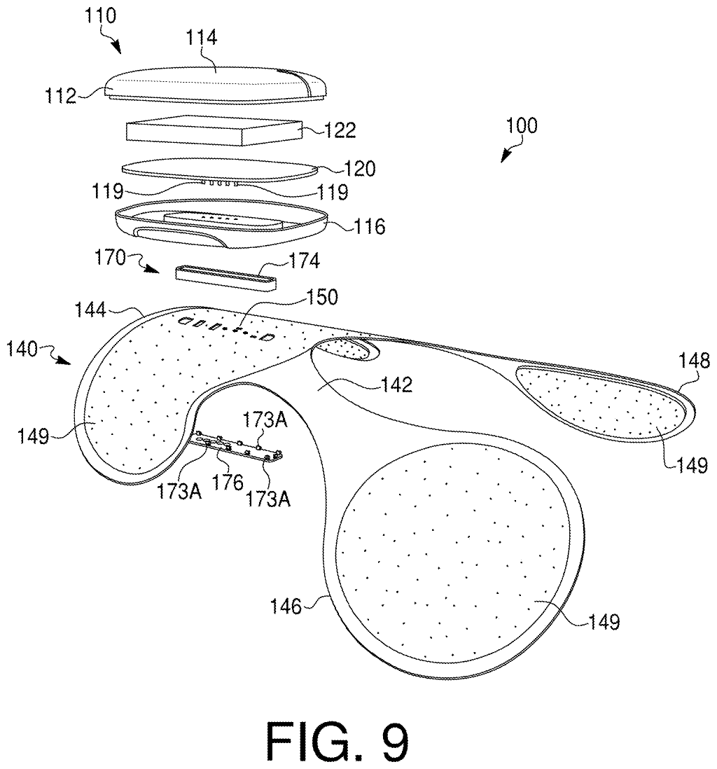

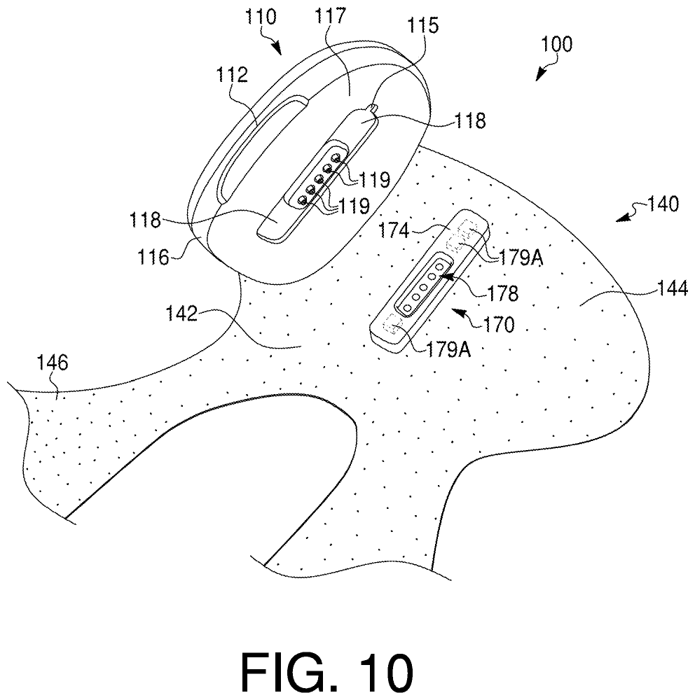

Referring now to FIG. 5, connector device 170 is depicted in a disassembled state with first wall 174 disengaged from second wall 176. In the example, connector device 170 may include one or more fastening mechanisms on first wall 174 and/or second wall 176 for maintaining housing 172 in an assembled state (FIG. 4). The one or more fastening mechanisms may include clips, protrusions, tabs, and/or various other suitable features for facilitating a selective attachment of first wall 174 and second wall 176. In some embodiments, as shown in FIG. 5, a fastening mechanism of second wall 176 may include an annular lip 173 that extends about a perimeter of second wall 176. Annular lip 173 may be configured to interface with a corresponding fastening mechanism of first wall 174, such as a recess (not shown) that is sized, shaped, and/or otherwise configured to mate with annular lip 173, thereby coupling second wall 176 to first wall 174. In other examples, a fastening mechanism of second wall 176 may include one or more clips 173A extending outwardly from the interior surface of second wall 176 for engaging an outer edge of first wall 174 (see FIGS. 8-9). As described in detail below, housing 172 may be disassembled when attaching connector device 170 to electrode device 140.

Connector device 170 may further include a second connection interface 177 that includes one or more apertures disposed along an interior surface of second wall 176. It should be appreciated that the number of apertures on second connection interface 177 may correspond to the number of apertures on first connection interface 178. Connector device 170 may include a circuit board 182 disposed within housing 172 between first wall 174 and second wall 176. Circuit board 182 may include charge port 180 and an electrode plate 184 that has one or more apertures 186. In some embodiments, electrode plate 184 may include a metal plate defining a metallic conductor that is configured to inductively and/or magnetically couple connector device 170 with stimulator device 110. For example, electrode plate 184 may be configured to change or induce a change in electrical current generated at stimulator device 110, thereby inducing a voltage (e.g., electrical current power transfer) at connector device 170, such as through electromagnetic induction. It should be appreciated that the number of apertures 186 on electrode plate 184 may correspond to the number of apertures on each of connection interfaces 177, 178. It should further be appreciated that the apertures of first connection interface 178, apertures 186 of electrode plate 184, and the apertures of second connection interface 177 may be aligned with one another when first wall 174 is attached to second wall 176 with circuit board 182 disposed therebetween.

Still referring to FIG. 5, connector device 170 may include one or more second cavities 171B along an interior surface of second wall 176 for receiving one or more second magnets 179B. The one or more second magnets 179B may be configured to mate with one or more first magnets 179A, thereby magnetically coupling first wall 174 to second wall 176 when housing 172 is fully assembled. Connector device 170 may further include one or more protrusions 175 extending outwardly from an interior surface of second wall 176, with a quantity of protrusions 175 corresponding to a quantity of apertures 185 positioned along circuit board 182 for receiving the one or more protrusions 175. The one of more protrusions 175 may be positioned along second wall 176 to define one or more connection points for coupling circuit board 182 to second wall 176, upon receipt within apertures 185. In other words, the one or more protrusions 175 may be configured to mate with the corresponding apertures 185 of circuit board 182 to reduce undesirable movement of circuit board 182 (and/or electrode plate 184 coupled thereto) relative to second wall 176.

Circuit board 182 may be any suitable circuit board, such as a printed circuit board (PCB), a rigid-flex circuit board, and more. Electrode plate 184, and particularly apertures 186, may be configured to interact with (e.g., receive) the plurality of pins 119 via the corresponding apertures on first connection interface 178, in response to stimulator device 110 connecting with connector device 170. For example, the one or more apertures 186 on electrode plate 184 may define electrical contacts that are configured to receive the plurality of pins 119 through first wall 174, and the corresponding apertures on second connection interface 177 may be configured to receive the plurality of pins 119 therein via apertures 186 on electrode plate 184. As described above, electrode plate 184 may define a power and/or pulse plate that is configured to establish an electrical connection between circuit board 182 and stimulator device 110 upon receiving the plurality of pins 119 within apertures 186. Accordingly, circuit board 182 may be operable to receive an electrical current or pulse transmitted from the plurality of pins 119 to electrode plate 184 while stimulator device 110 and connector device 170 are connected. Circuit board 182 and/or electrode plate 184 may be further configured to transfer said electrical current or pulse to electrode device 140 for delivery to the user.

Referring now to FIG. 6, electrode device 140 may include one or more electrode pads 149 positioned along various ends and/or portions of flexible body 142, such as on one or more of first end 144, second end 146, and third end 148. Each of the one or more electrode pads 149 may be configured to hold one or more electrodes proximal to a target treatment site (e.g., a muscle) on a user's body upon attaching electrode device 140 thereto. For example, electrode pads 149 (and/or an exterior surface of second wall 176) may include an outer adhesive layer and/or define an adhesive surface of flexible body 142 to facilitate attachment to the target treatment site. As described in detail herein, electrode pads 149 may be selectively attached to flexible body 142 and replaceable after use of electrode device 140. Electrode pads 149 may be made of various suitable materials, including but not limited to, a water-based electrode gel. In some embodiments, electrode pads 149 may be replaceable, solid electro-gel pads that are suitable for application and reapplication to the skin of a user's body without requiring additional moisturization or gel to facilitate attachment. In further embodiments, electrode pads 149 may include one or more fibers, such as a carbon fiber, which may facilitate in preserving a shape and/or flexibility of electrode pads 149.

Electrode device 140 may include one or more electrical connections 156 positioned on flexible body 142. Electrical connections 156 may include a wire and/or a cable that may be configured to electrically couple the one or more electrode pads 149 to connector device 170 and/or to one another. In some examples, electrical connections 156 may be covered, embedded, and/or mounted flush on flexible body 142 such that the wiring of electrical connections 156 may be fixed (e.g., adhered) to flexible body 142. For example, electrical connections 156 may be integrated within flexible body 142, such as woven into a fabric of flexible body 142. In another example, electrical connections 156 may be printed onto one or more surfaces of flexible body 142. In some examples, electrical connections 156 may not be visible and/or exposed from flexible body 142. It should be appreciated that electrode device 140, and particularly flexible body 142, may have various suitable shapes and/or sizes than those shown and described herein without departing from a scope of this disclosure. In some embodiments, a size and/or shape of flexible body 142 may be configured based on a target treatment site (e.g., a bodily region) on the user's body on which electrode device 140 is intended to be attached.

In the example, electrode device 140 may be an integrated, reusable, two-channel electrode and flexible body 142 may be selectively adjusted to multiple configurations, such as, for example, to conform flexible body 142 to a shape and/or arrangement of a target treatment site on a body of the user (e.g., long muscles vs. short muscles). Electrode device 140 may be configured to selectively attach to the target treatment site of a user's body along the exterior surfaces of electrode pads 149. As such, electrode device 140 may be operable for easy application and reapplication to the skin of a user's body. Electrode device 140, and particularly flexible body 142, may be flexible to promote an attachment of EMS system 100 to the user's body and a physical comfort to the user during movement while EMS system 100 is attached thereto. In some examples, electrode device 140 and/or components of electrode device 140, e.g., one or more of flexible body 142, electrode pads 149, and/or electrical connections 156, may be disposable.

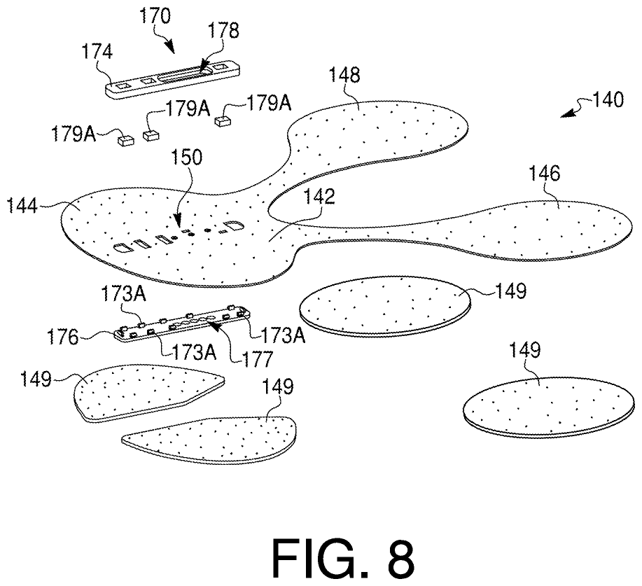

As shown in FIGS. 7-8, electrode device 140 may include one or more layers. It should be appreciated that the exemplary layers of electrode device 140 shown and described herein is merely illustrative such that electrode device 140 may include additional and/or fewer layers without departing from a scope of this disclosure. In the example, electrode device 140 may include flexible body 142 (e.g. a fabric) defining a first outer layer, and removable electrode pads 149 (e.g. hydrogel pads) defining a second outer layer that is opposite of the first outer layer. Electrode device 140 may include one or more interior layers disposed between the opposing outer layers. For example, electrode device 140 may include electrical connections 156 and a flexible printed circuitry 152 formed of a conductive material between the outer layers. Flexible printed circuitry 152 may serve as a lead (or multiple leads) between electrode pads 149 and stimulator device 110, when coupled thereto via connector device 170, such as via electrical connections 156. In some embodiments, the conductive material of flexible printed circuitry 152 may include a carbon “black” paper, a silver printed film, and/or a non-conductive substrate. In some embodiments, flexible printed circuitry 152 may include an ultraviolet-cured dielectric ink (e.g., DI-7510 dielectric ink).

In further embodiments, the conductive material of flexible printed circuitry 152 may include silver-printed traces on a carbon paper that may operable to reduce an electrical resistance that is generally provided by carbon paper alone. In other embodiments, the conductive material of flexible printed circuitry 152 may include a conductive ink printed on a woven polyester fabric (e.g., PS-606B Thermal Transfer, Black Woven Polyester). In this instance, the conductive ink may be printed on a woven fabric via any suitable method, including, e.g., thermal transfer, flexography, offset printing, or hot stamping.

Still referring to FIGS. 7-8, electrode device 140 may include one or more additional interior layers disposed between the opposing outer layers, such as an insulation film 154 serving as a protective insulating layer made from non-conductive materials (e.g., polyethylene terephthalate and/or woven polyester). Insulation film 154 may be configured to cover and/or insulate the user's body from the one or more conductive layers of electrode device 400, such as flexible printed circuitry 152.

Referring specifically to FIG. 8, electrode device 140 may include a connection interface 150 (e.g. electrical circuitry) along a portion of flexible body 142, such as along first end 144. Connection interface 150 may include one or more openings extending through flexible body 142, which may be configured to facilitate a connection (e.g., electrical, manual, etc.) between electrode device 140 and connector device 170. For example, flexible body 142 may be configured to receive first wall 174 along a first (top) surface of connection interface 150 and second wall 176 along a second (bottom) surface of connection interface 150. In the example, connector device 170 may include one or more fastening mechanisms 173A (e.g., clips) for attaching first wall 174 to second wall 176, and the one or more openings of connection interface 150 may be configured to receive fastening mechanisms 173A therethrough. Stated differently, connector device 170 may connect with electrode device 140 at connection interface 150 by attaching first wall 174 to second wall 176 (via the engagement of fastening mechanisms 173A through connection interface 150) with flexible body 142 disposed therebetween. Upon coupling connector device 170 to electrode device 140, connector device 170 may be configured to attach stimulator device 110 to electrode device 140 via a magnetic connection, as described above.

FIG. 9 depicts, for example, an exploded view of stimulator device 110, electrode device 140, and connector device 170 in a disassembled state. First wall 174 and second wall 176 of connector device 170 may engage electrode device 140 along opposing sides of flexible body 142 at connection interface 150, as described above. In this instance, the one or more fastening mechanisms 173A on second wall 176 may extend through the one or more openings on flexible body 142 at connection interface 150 to engage first wall 174 positioned along the opposing side of flexible body 142, thereby securing connector device 170 to electrode device 140. With connector device 170 attached to flexible body 142, the one or more magnets 179 (see FIG. 5) of connector device 170 may be configured to couple electrode device 140 to stimulator device 110 via a magnetic connection with magnets 126, 128 in housing 112 (see FIG. 2).

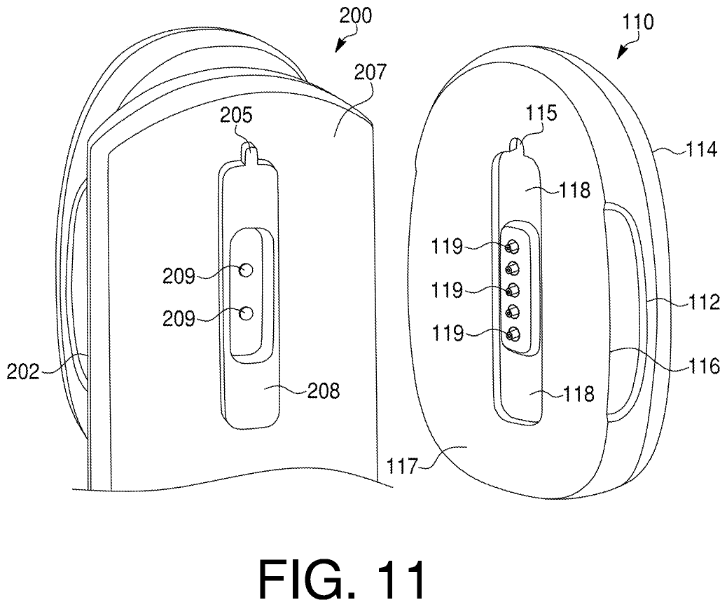

As best seen in FIG. 10, with connector device 170 attached to electrode device 140, first wall 174 may be configured to magnetically couple with the magnetic plate of bottom interface 117. In this instance, connection interface 178 may align with recess 118 and receive the plurality of pins 119 therein, thereby electrically coupling stimulator device 110 to electrode device 140 via connector device 170. As first connection interface 178 may be aligned with apertures 186 of electrode plate 184 (see FIG. 5), which mates with pins 119, connector device 170 may be configured to receive an electrical current or pulse generated by stimulator device 110 through the connection between pins 119 and electrode plate 184. It should be appreciated that stimulator devices 110 may be removably connected to electrode device 140 via connector device 170, and the connection may include a physical attachment (e.g., magnetic coupling) and an electrical connection for delivering electrical current and/or pulse to electrode device 140.

The relative strength of each magnet pairing between stimulator device 110 and connector device 170 may be sufficient to allow for a sturdy connection, while also allowing for selective disconnection between said devices when sufficient force is applied by a user. In some embodiments, as described above, the relative strength of each magnet in the respective devices of EMS system 100 may vary. For example, in some embodiments, one or more of first magnets 126 on stimulator device 110 may have a relatively stronger (or weaker) magnetic field strength (intensity) than second magnet 128. Further, one or more of magnets 179 on connector device 170 may also have varying magnetic field strength relative to one another, or may all have the same relative magnetic strength.

Still referring to FIG. 10, a physical connection between stimulator device 110 and connector device 170 may be sufficiently strong such that stimulator device 110 is inhibited from decoupling with connector device 170 when EMS system 100 is exposed to vibration or other bodily movements during use (e.g., during exercise, in therapy, etc.). In some embodiments, connector device 170 may be configured to facilitate ease in coupling stimulator device 110 to electrode device 140, such as to allow for connecting stimulator device 110 to electrode device 140 using one hand. In other embodiments, connector device 170 may be configured to wirelessly couple with stimulator device 110 via inductive and/or electromagnetic coupling, such that stimulator device 110 is not attached to connector device 170. In this instance, stimulator device 110 may be configured to wirelessly transfer electrical current (e.g., from generator 120) and/or communication (e.g., from controller 121) to connector device 170 when positioned within a proximity (e.g., a predefined distance) of connector device 170 and/or electrode device 140. By way of illustrative example only, the predefined distance may range from about 1 centimeter to about 305 centimeters, such as about 30 centimeters. It should be appreciated that the predefined distances described herein are merely exemplary such that stimulator device 110 and connector device 170 (and/or electrode device 140) may be wirelessly coupled to one another (e.g., via inductive coupling, electromagnetic coupling, etc.) at other suitable distances.

In some embodiments, connector device 170 may include one or more sensors (not shown) disposed within and/or positioned along an exterior of housing 172. The one or more sensors may be configured to detect one or more operating characteristics of stimulator device 110 and/or electrode device 140. In the example, the one or more sensors may include Hall effect sensors configured to detect when an electrical connection is made with connector device 170 (e.g., via connection interface 178, charge port 180, etc.), and/or a type of electrical connection made with connector device 170. For example, a Hall effect sensor of connector device 170 may be configured to detect when stimulator device 110 may be connected thereto, and thereby in electrical communication with electrode device 140. In some embodiments, connector device 170 may be configured to generate a user feedback, such as generating a light to be displayed on stimulator device 110 (e.g., at indicator 111) upon detecting the electrical connection. In other embodiments, connector device 170 may be configured to generate a user feedback on electrode device 140.

In further embodiments, a sensor of connector device 170 may be configured to detect when stimulator device 110 is connected to a charger device, and thereby generate a user feedback in response. When connected to electrode device 140 via connector device 170, indicator 111 on stimulator device 110 may be activated and visible to a user of EMS system 100, so as to be readily detectable and monitored during use of EMS system 100. In this instance, EMS system 100 may be configured to generate a visual user feedback during use. It should be appreciated that, in other embodiments, EMS system 100 may be configured to generate various other user feedbacks indicating an active operation of EMS system 100 to the user, including but not limited to, an audible and/or tactile feedback.

Referring now to FIG. 11, an exemplary charger device 200 of EMS system 100 is depicted. Charging device 200 may include a housing 202 having a bottom surface 207 and a protrusion 208 positioned along bottom surface 207. Protrusion 208 may be sized, shaped, and/or otherwise configured to mate with recess 118 of stimulator device 110, thereby facilitating a connection between stimulator device 110 and charger device 200. In some embodiments, protrusion 208 may include an alignment feature 205 that is configured to mate with alignment feature 115 on bottom interface 117 of stimulator device 110, thereby facilitating a proper alignment between the devices when coupling with one another.

Charger device 200 may further include one or more power plates 209 positioned along bottom surface 207, and particularly on protrusion 208. Upon coupling stimulator device 110 to charger device 200 via a physical connection between protrusion 208 and recess 118, the one or more power plates 209 may be configured to interface with the one or more pins 119. In this instance, the one or more power plates 209 may be configured to establish an electrical connection between a battery (not shown) of charger device 200 and battery 122 of stimulator device 110 (see FIG. 2). The electrical connection between charger device 200 and stimulator device 110 may allow for a transfer of electrical charge from the battery of charger device 200 to battery 122 of stimulator device 110 when stimulator device 110 is decoupled from connector device 170. In some embodiments, charger device 200 may be configured to connect stimulator device 110 to an external power source (e.g., an electrical outlet, a USB power source, etc.) to charge battery 122. In other embodiments, charger device 200 may be configured to charge multiple stimulator devices 110 simultaneously. In some embodiments, charger device 200 may be configured to couple with connector device 170, such that charger device 200 is electrically coupled to stimulator device 110 via connector device 170.

Still referring to FIG. 11, charger device 200 may be configured to monitor and detect one or more parameters, such as a connection to stimulator device 110, when stimulator device 110 is being actively charged, and/or when stimulator device 110 may be fully charged. Charger device 200 may be sized and/or shaped to have a relatively lightweight configuration (e.g., less than about 400 grams, less than about 500 grams, or less than about 600 grams). In some embodiments, charger device 200 may be formed of a sturdy, a shatterproof, and/or a shatter-resistant material (e.g., plastic). Charger device 200 may further include one or more labels and/or markings that display information indicative of the contents and/or power capabilities of charger device 200 (e.g., a lot number, a voltage capacity, a power usage, a connection type, and the like).

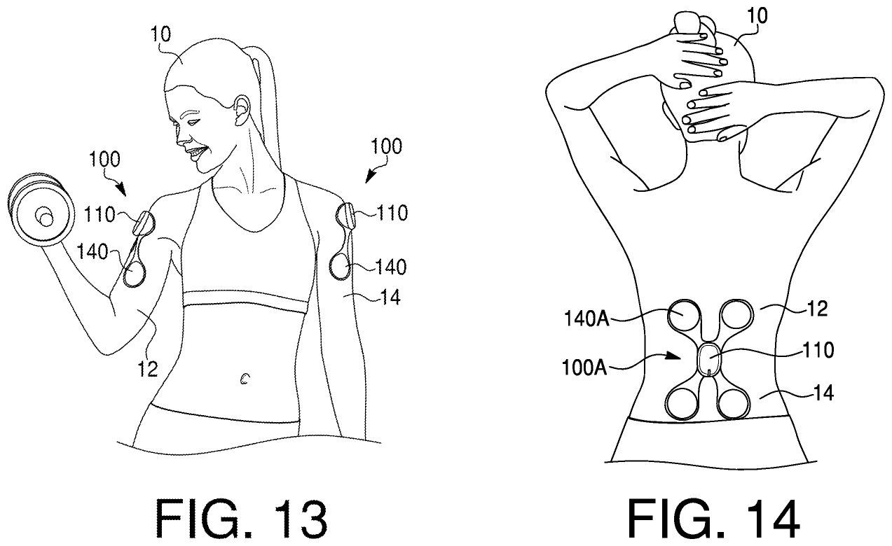

FIG. 12 depicts an exemplary kit or carrying case 300 for storing and/or transporting the one or more devices of EMS system 100. Carrying case 300 may include a first sleeve 302 and a second sleeve 304, which may be selectively connected to one another by a fastening feature to enclose carrying case 300 with EMS system 100 stored therein. The fastening feature may include, for example, a zipper, a hook-and-loop fastener, a clip, and more. Each of first sleeve 302 and second sleeve 304 may be formed of a material configured to shield and/or protect the devices of EMS system 100 therein when in a closed state. Carrying case 300 may include one or more cavities 306 positioned along an interior surface of first sleeve 302, each of which may be sized, shaped, and/or otherwise configured to house one or more stimulator devices 110. Carrying case 300 may further include a pocket 308 that is at least partially enclosed by a wall 310 on the interior surface of first sleeve 302. Pocket 308 may be sized, shaped, and/or otherwise configured to house one or more devices therein, such as, for example, connector device 170, a charging cable 190, and more. In the example, wall 310 may include a mesh pouch configured to maintain the one or more devices within pocket 308.

Carrying case 300 may include a compartment 312 defined within an interior surface of second sleeve 304, which may be sized, shaped, and/or otherwise configured to house electrode device 140. Compartment 312 may be enclosed by a movable panel 314 configured to maintain electrode device 140 within compartment 312. In some embodiments, movable panel 314 may be an electrode backing film, such that electrode device 140 may be adhered to the electrode backing of movable panel 314 while housed in carrying case 300 within second sleeve 304. In some embodiments, compartment 312 may include one or more removable protective layers (e.g., a removable plastic film) configured to cover and protect electrode pads 149 when electrode device 140 is stored therein. It should be appreciated that carrying case 300 may include a bespoke designed package that is reusable and configured to protect aspects of EMS system 100 (e.g., one or more stimulator devices 110, electrode devices 140, connector devices 170, and more) from active environments (e.g., therapeutic centers, hospitals, rehabilitation facilities, gyms, etc.) and to prevent exposure of one or more components of electrode device 140 (e.g., electrode pads 149) to air.

It should further be appreciated that EMS system 100, and particularly one or more of stimulator device 110, electrode device 140, and/or connector device 170, may be configured and operable for a variety of storage, transport, and operating conditions in accordance with various regulatory standards, including but not limited to, the International Electrotechnical Commission standard. For example, EMS system 100 may be suitable for storage and transport at temperatures of between about −20° C. and about 75° C., such as about −25° C. and about 70° C. Further, EMS system 100 may be suitable for storage and transport at a non-condensing relative humidity of up to about 93%. EMS system 100 may be operable at temperatures of between about 5° C. and about 40° C., at non-condensing relative humidities between about 15% and about 93%, and at atmospheric pressures between about 700 hPa to about 1060 hPa. It should be understood that the exemplary storage, transport, and operating conditions of EMS system 100 described herein are merely illustrative and in accordance with exemplary regulatory standards, such as the International Electrotechnical Commission standard.

Each of the one or more devices of EMS system 100 may have various lifespans, and may be replaceable either separately or as a group. For example, in some exemplary embodiments, EMS system 100 may be configured for use between about 1 and 2 hours per day, and up to about 5 days per week. At such a level of use, stimulator device 110 may require battery 122 to be charged once daily. Stimulator device 110 may have a lifespan ranging from about 1 year to about 7 years. In some embodiments, charger device 200 and/or connector device 170 may have similar lifespans as stimulator device 110. Electrode device 140, and particularly flexible body 142, may have a lifespan ranging from about 1 month to about 8 months, and electrode pads 149 may have a lifespan ranging from about 1 day to about 10 days.

EMS system 100 according to the present disclosure may, in some embodiments, be manufactured according to regulatory standards. For example, in some embodiments, EMS system 100 may be designed and manufactured under conditions specified by the International Organization for Standardization (ISO) (e.g., ISO 13485) of the International Electrotechnical Commission (IEC). As described above, EMS system 100 may include device packaging, labelling, instructions for use, and contraindications in accordance with one or more regulatory standards. Stated differently, each of the one or more devices of EMS system 100 may be configured and operable in accordance with certain regulatory standards. Standards for EMS system 100 may include safety standards, environmental standards, standard for co-existence with other electronic devices, system maintenance standards, labelling and packaging standards, and/or standards for instructions for use. As further examples, one or more devices of EMS system 100, such as stimulator device 110, may be configured and operable to satisfy certain tests, such as shock tests, drop tests, and more.

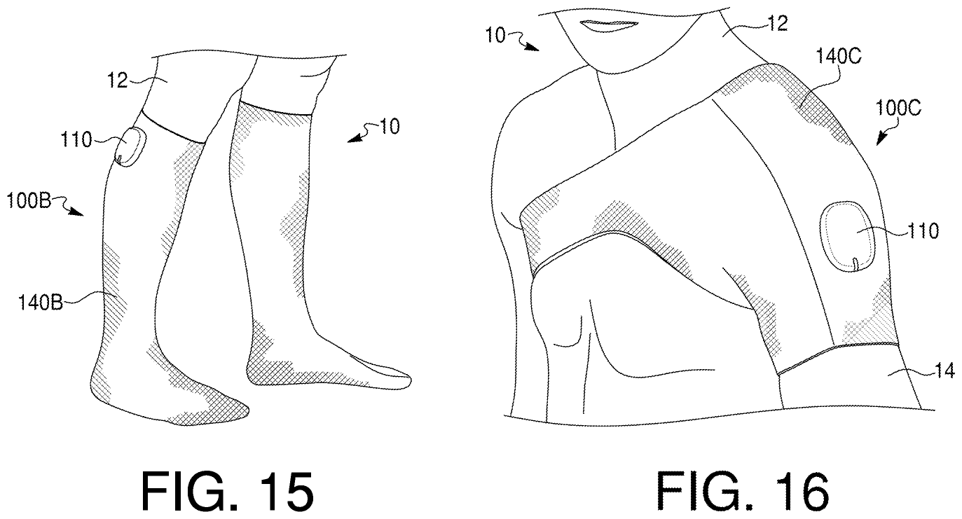

FIGS. 13-16 depict various embodiments and exemplary applications of EMS system 100 onto a user's body. For example, FIG. 13 depicts a pair of EMS systems 100 attached to a first bodily region 12 (e.g., a first arm) and a second bodily region 14 (e.g., a second arm) of a user 10, respectively. Electrode device 140 may be generally V-shaped, with flexible body 142 adhering to a curvature and/or a shape of the respective bodily regions 12, 14. Although EMS systems 100 are shown and described herein as being attached to one or more arms of the user 10, it should be appreciated that the first bodily region 12 and the second bodily region 14 are merely illustrative such that EMS system(s) 100 may be secured to various other regions and/or areas of the body of user 10 (e.g., a head, a neck, a torso, a waist, a leg, a foot, etc.). Further, it should be appreciated that additional and/or fewer EMS systems 100 may be simultaneously attached to a body of user 10 than those shown and described herein without departing from a scope of this disclosure.

By way of further example, FIG. 14 depicts another exemplary EMS system 100A having an electrode device 140A with a generally X-shaped body adhered to a body of user 10. In this instance, electrode device 140A may extend across a first bodily region 12 (e.g., a center back) and a second bodily region 14 (e.g. a lower back) of user 10. Due to the generally X-shaped configuration of electrode device 140A, EMS system 100A may securely adhere to a relatively larger surface area of the body relative to EMS system 100. It should be appreciated that FIGS. 13 and 14 depict exemplary embodiments of an EMS system having varying sizes, shapes, and/or configurations, such as different electrode devices with different shapes. It should be understood that electrode devices 140, 140A may have various other suitable sizes, shapes, and/or configurations than those shown and described herein without departing from a scope of this disclosure.

In some embodiments, EMS system 100 may be integrated with one or more articles, including but not limited to, types of apparel, garments, physical therapy devices, medical equipment, and more. It may be contemplated that one or more devices of EMS system 100 may be integrated into and/or coupled with said articles, such as stimulator device 110, electrode device 140, and/or connector device 170. By way of illustrative example only, such articles may include, but are not limited to, bandages, slings, compression socks, arm/wrist bands, belts, gloves, sleeves, shirts, and more. By integrating and/or coupling one or more devices of EMS system 100 with said articles, the need for a separate application of EMS system 100 and a supplemental therapy device onto a user's body may be reduced.

FIG. 15 depicts an exemplary EMS system 100B with stimulator device 110 coupled to a first user apparel 140B. First user apparel 140B may include various garments, such as a compression sock. User 10 may wear first user apparel 140B on a first bodily region 12, e.g., a lower leg. In some embodiments, first user apparel 140B may include one or more layers as described in detail above with respect to electrode device 140 (see FIGS. 7-8). Accordingly, first user apparel 140B may be configured and operable like electrode device 140. For example, first user apparel 140B may be formed of a conductive material(s) that may be configured to transfer and/or distribute the electrical current or pulse generated by stimulator device 110 across first bodily region 12. In some examples, stimulator device 110 may be coupled to first user apparel 140B via connector device 170 positioned therebetween.

FIG. 16 depicts another exemplary EMS system 100C with stimulator device 110 coupled to a second user apparel 140C. Second user apparel 140C may include various suitable garments, such as a brace. User 10 may wear second user apparel 140C on a first bodily region 12 (e.g., a shoulder), such that second user apparel 140C may be sized, shaped, and/or otherwise configured to extend towards a second bodily region 14 (e.g., an arm) of user 10. Second user apparel 140C may be configured to transmit the electrical current or pulse generated by stimulator device 110 to first bodily region 12 and second bodily region 14 during use of EMS system 100C. It should be appreciated that an EMS system of the present disclosure may include a plurality of user apparels, such that user 10 may utilize the plurality of user apparels (e.g., first user apparel 140B, second user apparel 140C, etc.) simultaneously along multiple bodily regions.

EMS systems 100, 100B, 100C may be operably compatible for use with an exemplary software application and/or exemplary user interface. In some embodiments, EMS system 100 (and particularly electrode device 140) may include a plurality of leads as described in detail above, such that the plurality of leads may be selectively controlled by said software application and/or user interface, such as through stimulator device 110, and particularly stimulator mechanism 113 (e.g., controller 121). In other words, the software application and/or user interface may be configured to activate EMS systems 100, and particularly the plurality of electrodes and/or electrode leads of EMS system 100 (e.g., electrode pads 149), for controlling delivery of electrical muscle stimulation to a user's body. In some embodiments, the software application and/or user interface may be accessible and/or operational from an external electronic device that is communicatively coupled to EMS system 100, and particularly to stimulator device 110. The external electronic device may include, for example, a mobile phone, a tablet, a computer, a cloud computer system, a wearable device, and more). The software application may include, for example, a mobile device application or set of software applications that may allow a user to interact with EMS system 100 in a variety of ways.

In some embodiments, a user may download, program, edit, revise, select, and/or share exercise programs, stimulation patterns, short-term and long-term EMS regimens, and the like using the software application and/or user interface. For example, a user may configure EMS-related exercises by transmitting user inputs to stimulator device 110 via the user interface. The user interface may include a display screen that is operable to display, for example, a human body muscle diagram, on which a user may select the muscle group(s) that the user wishes to stimulate with EMS system 100 for various purposes, e.g., healing, rehabilitation, warm-up, training, and/or recovery. Such selection may be through, for example, a touch-screen or mouse selection on the user interface. The software application and/or user interface may be configured to display a variety of stimulation programs and/or patterns pertaining to the selected muscle group(s) for user selection.

Software applications and/or user interfaces may be configured to facilitate a creation, a revision, and/or a distribution of various stimulation patterns and/or exercise programs among a plurality of users, user groups, and/or communities (e.g., medical, recreational, etc.). For example, a physical therapist or other medical professional may be able to recommend one or more exercise programs and/or stimulation patterns to a patient by sharing such a program to a patient's software application and/or user interface. Stimulation patterns and/or exercise programs may be made available for download onto the user's external electronic device. Additionally, the software application and/or user interface may be equipped with systems into which users may log in (e.g., to the software application, user interface, and/or to a remote server or system connected to the software application or user interface) and create, access, and/or share exercise programs and/or stimulation patterns on a user account.

By way of further example, exemplary software applications and/or user interfaces may be capable of collecting and recording data about the user's use of one or more EMS systems 100, either locally to the user's personal external device (e.g., a mobile phone, a tablet, a computer, or other electronic device), or to a remote storage system (e.g., a remote server, a cloud database, or other computer). In some embodiments, the software application and/or user interface may be configured to accept an instruction to record usage data for the user's EMS system 100 over a given period of time, and associate such data with a profile (e.g., a medical profile, a login profile, or other identifying profile) of the user. Additionally, the exemplary software applications and/or user interfaces may be configured to monitor and/or limit usage of one or more EMS systems 100 by the user for various safety concerns, such as by monitoring and/or limiting an intensity and/or duration of electrical stimulation generated by the one or more EMS systems 100.

An example of some aspects of the software application and/or user interface for use on an external electronic device, such as a mobile phone (e.g., StimRay application software) and its interaction with one or more EMS systems 100 may include wireless communication via a personal area network (PAN) technology designed for small, low power devices such as Bluetooth Low Energy (BLE) (e.g., Bluetooth 4.0, Bluetooth SMART). It should be appreciated that BLE may allow devices to provide data and services to connected host devices.

Once the software application, e.g., via the external electronic device, is wirelessly paired to one or more EMS systems 100, the software application may be operable to control and/or monitor the one or more EMS systems 100 during use. For example, the software application may include a predetermined pattern for controlling EMS system(s) 100, such as a strength, a frequency, and/or a duration of the electrical current or pulses generated by each EMS system 100. The stimulation pattern may be initiated by a user activating EMS system 100 via the user interface, such as by actuating a “Start” button within the software application. Once started, the software application or user interface may be selectively actuated to pause, stop, and/or reactivate the electrical stimulation generated by EMS system 100 by the user. In some embodiments, the software application may be configured to automatically (e.g., autonomously) operate EMS system 100 in accordance with one or more preset or user-defined settings, such as a warm up phase, an exercise phase, and/or a cool down phase.

A duration of said operation phases may be based on various factors, such as a timer and/or a user input (e.g., actuating a “Stop” or “Pause” button within the software application). By way of further example, the software application may allow a user to stop or pause the timer during the warm up phase, exercise phase, and/or cool down phase, and reactivate the timer in response to a corresponding user input. In some embodiments, the software application may be operable to identify faults in EMS system 100 and automatically pause or stop continued operation of EMS system 100, to inhibit injury to the user. For example, a short in EMS system 100 circuitry may be communicated to the software application, thereby causing the software application to pause or stop EMS system 100. This automatic operation may help minimize injury or discomfort to the user during use.

EMS system 100 may be configured and operable to implement various safety measures to ensure proper use and performance of EMS system 100. For example, the one or more devices of EMS system 100 (e.g., stimulator device 110) may be configured to limit an intensity and/or duration of generated electrical stimulation for delivery to the user. Further, the exemplary software application and/or user interface that is communicatively coupled to EMS systems 100 may be configured and operable to only be accessible and/or controllable via devices authorized by the user. In some embodiments, EMS system 100 may implement security mechanisms such as only permitting wireless (e.g., Bluetooth) connection to said authorized devices. Upon connecting EMS system 100 to said device, all communications between the authorized device and EMS system 100 may be authenticated and encrypted. As an illustrative example, wireless communications between the external electronic device and EMS system 100 may occur over BLE (e.g., Bluetooth 4.0 or Bluetooth SMART) using an application-side framework (e.g., Apple's Core Bluetooth Framework).

EMS system 100, and the exemplary software application and/or user interface providing access control to EMS system 100, may improve the ease in delivering electrical stimulation to a user's body (e.g., muscles) by allowing the user to selectively position EMS system 100 at the target treatment sites and easily manipulate operation of EMS system 100 at an external device. Such operation characteristics may enhance a treatment and/or recovery impact for the user given that EMS system 100 may be utilized by users who are immobilized and/or actively engaged in an activity (e.g., exercise) by facilitating control of EMS system 100 through the external device.

The exemplary hardware and software of EMS system 100 described above may increase a robustness and reliability for the user. Generally, Bluetooth devices may be difficult to configure and manage, and may be prone to unreliability with respect to connectivity between multiple devices. As EMS system 100 may be used during another primary activity (e.g., exercising), the software application may be configured to require minimal guidance from users to ensure continued operation in noisy radio environments. The design of the hardware and software of EMS system 100 may take this requirement into account. For example, by preconfiguring exercise session steps in the software and transferring these steps to the hardware (e.g., stimulator device 110), EMS system 100 may be operable to automatically continue to operate in an environment with frequent but short connectivity challenges. The software application may be configured to manage connectivity with bonded external devices automatically, which may minimize the impact that breaks in connectivity may have on the user experience.

EMS system 100 and the corresponding software application may improve battery life performance by parsimoniously managing data transfer among the external electronic device and EMS system 100, which may minimize active radio time. The software application may be further operable to operate EMS system 100 in the background of the external electronic device's operating system in an asynchronous manner, thereby minimizing an impact on a battery life of said device. The software application may be configured to perform a plurality of operations for controlling EMS system 100, such as reading user profile information from an application, requesting user permission to read user biometric data (e.g., pulse data), and more. The software application may be further configured to manage and update user operation preferences of EMS system 100 on startup and/or shutdown, accept and/or manage user inputs for controlling EMS system 100, communicate with EMS system 100 (e.g., via Bluetooth) for command control, and/or monitoring or reading exercise parameters for controlling an extent of electrical stimulation generated by EMS system 100.

EMS systems 100 may be suitable for various uses, including but not limited to, prescriptive use, over-the-counter sale, and more. As described above, EMS systems 100 may be used in a variety of ways, including but not limited to, use in a home, a doctor's office, a hospital, a medical care facility, an athletic training facility, and more. EMS system 100 may be suitable for use by a variety of users, including but not limited to, healthy, able-bodied individuals and/or individuals undergoing treatment to rehabilitate and/or strengthen muscles in their bodies. EMS system 100 may be configured and operable to stimulate a wide variety of muscle groups in a user's body, including but not limited to, abdominal muscles, upper arm muscles (e.g., biceps and triceps), shoulder muscles, gluteal muscles, lateral muscles, lower back muscles, trapezius muscles, abductors, calf muscles, forearm muscles, hamstrings, quadriceps, pectoral muscles, and more. EMS system 100 may be configured for use in physical therapy, rehabilitation, athletic training, and/or to prevent medical conditions or illnesses (e.g., pressure ulcers or deep vein thrombosis).

In some embodiments, multiple EMS systems 100 may be used simultaneously on a user's body to stimulate different muscles (see FIG. 13). The multiple EMS systems 100 may be controlled via a single software application and/or user interface. For example, in some embodiments, two or more EMS systems 100 may perform coordinated electrical stimulation on two or more different parts of the user's body (e.g., mirror-image stimulation on two or more sides of the user's body). In such embodiments, multiple EMS systems 100 may be communicatively linked together in a network (e.g., a mesh network via a wireless connection) to stimulate multiple muscles of a user in a user-defined and/or predetermined pattern. For example, multiple EMS systems 100 may be coordinated in a mesh network to create and/or monitor electrical stimulation of a user's legs, to simulate a corresponding movement of the legs (e.g., walking, running, or other muscle usage), and more. Such coordination may be controlled by the single software application and/or user interface linked to the multiple EMS systems 100. In further embodiments, multiple stimulator devices 110 in a linked network may be attached to a single, larger electrode device 140 having multiple leads, to create and/or monitor muscle stimulation in a given pattern.

In some embodiments, the software application may be configured and operable to detect, record, and collect user stimulation data from multiple EMS systems 100, each having one or more stimulator devices 110. The data gathered from the multiple EMS systems 100 may be used by the software application to generate complex stimulation patterns, to create or modify stimulation patterns based on a larger data set, and/or to serve as a basis for machine learning. For example, data gathered from multiple EMS systems 100 (or multiple stimulator devices 110) may, over time, show trends as to the safety and/or effectiveness of the stimulation patterns to treating the user's body. Such user stimulation data may be used by the software application to generate new, improved stimulation patterns, or update existing stimulation patterns, that are unique and personalized to the user. Such data may also be used by the software application to inform training and/or therapy decisions for the user. Also, data may be distributed to multiple EMS systems 100 to distribute stimulation patterns, update stimulation patterns, update safety protocols, etc. In some examples, the software application may be configured such that control and delivery of electromagnetic pulses may be automated based on other suitable parameters, e.g., application of one or more EMS systems 100 to one or more regions along a user′ body, an intended use of EMS system 100 (e.g., treatment, rehabilitation, recreational, etc.) on a particular body part, a desired outcome from the stimulation session, etc.

In exemplary use, EMS system 100 may be operated by downloading and/or installing a software application on an external electronic device, such as a mobile phone, a tablet, a computer, a wearable device, etc. Stimulator device 110 may be charged prior to use of EMS system 100 as shown and described above, such as by charger device 200 (see FIG. 11), charging cable 190 (e.g., a micro-USB) (see FIG. 12), or any other suitable method. One or more electrode devices 140 may be secured to a user's body, with electrode pads 149 applied to a user's skin at one or more muscle groups that are targeted for electrical stimulation (see FIGS. 13-14). One or more stimulator devices 110 may be connected to the one or more electrode devices 140 via a corresponding connector device 170.

In this instance, the software application and/or user interface of the external electronic device may detect and register the one or more stimulator device(s) 110, such as by one or more sensors as discussed in detail above. It should be understood that registration between the software application and the one or more stimulator devices 110 may be established before or after stimulator device(s) 110 are coupled to electrode device 140, which is secured to the user's body, or at any other suitable point during use of EMS system 100. The software application may automatically (e.g., autonomously) control the delivery of electromagnetic current or pulse to the targeted muscle groups, such as in response to a user selecting and/or configuring a stimulation mode (e.g., a preset mode, a user-defined mode, etc.). Alternatively, the user may actively control the delivery of electromagnetic current or pulse to the targeted muscle groups, such as in response to the user interface (e.g. a button, a touchscreen, etc.) of the external electronic device receiving one or more user inputs. Such user inputs may include setting and/or adjusting an operating command (e.g., initiating, pausing, and/or ceasing stimulation), an operating parameter (e.g., an intensity, a duration, or a frequency of stimulation), and more.

The present disclosure is not limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s). Wherever possible, the same reference numbers are used throughout the drawings to refer to the same or like parts. While the disclosed methods, devices, and systems are described with exemplary reference to an electrical muscle stimulation (EMS) system, it should be appreciated that the disclosed embodiments may be applicable to any electrical stimulation system, such as neuromuscular stimulation. Also, the disclosed embodiments may be applicable to any type of stimulator or electrical stimulation device.

It should be appreciated that in the above description of exemplary embodiments, various features of the invention are sometimes grouped together in a single embodiment, figure, or description thereof for the purpose of streamlining the disclosure and aiding in the understanding of one or more of the various inventive aspects. This method of disclosure, however, may not be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments may be used in any combination. Thus, while certain embodiments have been described, other and further modifications may be made thereto without departing from invention scope of this disclosure, and it is intended to claim all such changes and modifications. Each aspect of the present invention may be added or deleted to customize the system to a user's preference.

1. An electrical stimulation system, comprising:
a stimulator including a generator and a controller, the generator is configured to generate an electrical current and the controller is configured to receive a user input for activating the generator;
an electrode including a flexible body and one or more electrode pads positioned along the flexible body; and
a connector configured to extend through the flexible body and electrically couple with the stimulator by inductive coupling, wherein the connector is configured to electrically couple the generator to the one or more electrode pads in response to the stimulator being positioned within a predefined distance to the connector, such that the controller is configured to wirelessly transmit the electrical current from the generator to the one or more electrode pads via the connector in response to the user input.

2. The electrical stimulation system of claim 1, wherein the predefined distance includes about 1 centimeter to about 30 centimeters.

3. The electrical stimulation system of claim 1, wherein the inductive coupling between the connector and the stimulator includes an electromagnetic connection;
wherein the connector is configured to wirelessly transmit the electrical current from the generator to the connector, and the connector is configured to distribute the electrical current to the one or more electrode pads, and
wherein the stimulator includes one or more magnets and the connector includes one or more corresponding magnets that are configured to mate with the one or more magnets of the stimulator, thereby magnetically coupling the stimulator to the connector.

4-5. (canceled)

6. The electrical stimulation system of claim 1, wherein the connector has a housing that is configured to receive at least a portion of the flexible body, thereby coupling the electrode to the connector.

7. The electrical stimulation system of claim 1, wherein the controller is configured to determine a speed, a duration, an intensity, or a frequency of the electrical current generated by the generator.

8. The electrical stimulation system of claim 1, wherein the flexible body includes a main portion and one or more legs extending outwardly from the main portion, wherein the connector is configured to extend through the flexible body at the main portion, and each of the one or more legs includes at least one of the one or more electrode pads.

9. The electrical stimulation system of claim 1, wherein the one or more electrode pads are selectively coupled to the flexible body, and configured to attach the electrode to a body of a user; and
wherein the one or more electrode pads define an adhesive interface of the flexible body that is configured to couple the electrode to the body of the user.

10. (canceled)

11. The electrical stimulation system of claim 1, wherein the electrode includes one or more wires enclosed or embedded within the flexible body, the one or more wires are configured to electrically couple the one or more electrode pads with the connector; and
wherein the flexible body includes a connection interface defining one or more openings, the connection interface is configured to receive at least a portion of the connector through the one or more openings.

12. (canceled)

13. The electrical stimulation system of claim 1, wherein the connector includes a housing defined by a first wall and a second wall;
wherein at least a portion of the electrode is received within the connector with the flexible body positioned between the first wall and the second wall, thereby coupling the connector to the electrode; and
wherein one or more of the first wall and the second wall includes a fastening mechanism that is configured to extend through the flexible body when the electrode is received between the first wall and the second wall.

14. (canceled)

15. The electrical stimulation system of claim 1, wherein the connector includes one or more sensors configured to detect one or more operating characteristics of the stimulator or the electrode; and
wherein the controller is configured to activate the generator in response to the one or more sensors detecting an electrical connection between the stimulator and the electrode.

16. (canceled)

17. The electrical stimulation system of claim 1, wherein the stimulator includes a housing formed of a biocompatible material, a water-resistant material, a shatter-resistant material, or a shock-resistant material;
wherein the stimulator includes an indicator positioned along an exterior surface of the housing that is configured to generate a user feedback indicative of an operation status of the stimulator or the electrode, and at least one rechargeable battery that is configured to supply electrical power to one or more of the generator and the controller; and
wherein the stimulator is communicatively coupled to at least one external electronic device, and the controller is configured to receive the user input for operating the generator from the at least one external electronic device.

18-20. (canceled)

21. The electrical stimulation system of claim 1, wherein the generator is configured to generate an electrical waveform over a wide range of signals, and to convert the electrical waveform to one or more pulses of the electrical current.

22. (canceled)

23. A method for operating an electrical stimulation system, the electrical stimulation system comprising a stimulator, a connector, and an electrode, wherein the stimulator is electromagnetically coupled to the connector and the electrode is at least partially received within an interior of the connector, such that the connector is configured to electrically couple the stimulator to the electrode;
the method comprising:
attaching the electrode to a body of a user such that the electrode is in contact with the body;
activating the stimulator to generate an electrical pulse that is transferred to the connector; and
delivering the electrical pulse from the stimulator to the electrode via the connector, thereby stimulating a portion of the body that is in direct contact with the electrode.

24. The method of claim 23, wherein the electrical stimulation system is communicatively coupled with an external electronic device, and the stimulator is configured to receive a user input from the external electronic device for activating the stimulator; and
wherein the external electronic device includes a software application that is communicatively coupled with the stimulator a plurality of electrical stimulation systems, such that the user input is generated at the software application and transmitted to the stimulator.

25. (canceled)

26. The method of claim 24,
wherein the user input defines one or more parameters of the electrical pulse including a speed, a duration, an intensity, or a frequency of the electrical pulse generated by the generator.

27. (canceled)

28. The method of claim 23, further comprising at least one sensor that is configured to detect an electrical connection between the stimulator and the electrode; the method comprising:
activating the stimulator automatically in response to the at least one sensor detecting the electrical connection between the stimulator and the electrode.

29. The method of claim 23,
wherein the stimulator includes one or more conductive pins configured to electrically couple with a control board of the connector, such that the control board is in electrical communication with the generator and the controller.

30. The method of claim 29, wherein the connector includes an electrode plate that is in electrical communication with the control board and the one or more electrode pads, the electrode plate is configured to receive the one or more conductive pins, thereby electrically coupling the electrode to the stimulator via the connector; and
wherein the connector is configured to transmit the electrical current generated by the generator to the one or more electrode pads via a connection between the electrode plate and each of the one or more conductive pins and the one or more electrode pads.

31. (canceled)

32. A computer-implemented method for operating an electrical stimulation system, the electrical stimulation system including a stimulator, a connector, and an electrode, wherein the stimulator includes a conductive pin, the electrode includes an electrode pad, and the connector includes a power plate that is configured to electrically couple the conductive pin with the electrode pad; the method comprising:
connecting a user device with the stimulator, such that the user device is in communication with the stimulator;
generating an electrical current with the stimulator in response to the stimulator receiving at least one input from the user device wherein the electrical current is transmitted to the connector via connection between the conductive pin and the power plate; and
delivering the electrical current from the electrode pad in response to the electrode receiving the electrical current from the connector, wherein the electrical current is transmitted to the electrode via connection between the power plate and the electrode pad.

33. The computer-implemented method of claim 32, wherein the user device is wirelessly connected to the stimulator via Bluetooth Low Energy or Wi-Fi.
